(19) United States
(12) Patent Application Publication
(10) Pub. No.: US 2005/0113396 A1
(43) Pub. Date: May 26, 2005

(54) 2-Guanidino-4-heterocyclylquinazolines

(76) Inventors: Rolf Gericke, Seeheim-Jugenheim (DE); Norbert Beier, Reinheim (DE); Claudia Wilm, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/498,806

(22) PCT Filed: Nov. 21, 2002
(86) PCT No.: PCT/EP02/13061

(30) Foreign Application Priority Data
Dec. 15, 2001 (DE) 101 61 767.4

(51) Int. Cl. A61K 31/517; C07D 43/04

(52) U.S. Cl. 514/266.2; 544/284

(57) ABSTRACT

The invention relates to compounds of the formula (I): in which (Y) is formula (I) or formula (III) and Het, R¹, R², R⁵, R⁶, R⁷ and R⁸ are as defined above, and salts and solvates thereof, and to the use thereof as NHE-3 inhibitors.

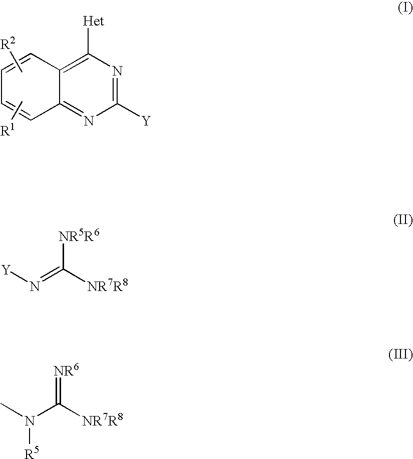

The invention relates to compounds of the formula I 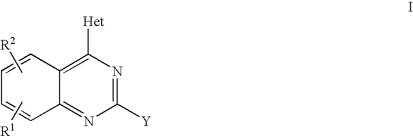
in which 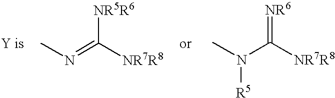
Het is a saturated, unsaturated or aromatic heterocyclic radical which is unsubstituted or monosubstituted or polysubstituted by R³ and/or R⁴,
R¹, R² R³
and R⁴ are each, independently of one another, H, A, OA, Hal, CF₃, CH₂CONH₂, CH₂CO₂H, CH₂CO₂A, CH₂NH₂, CH₂NA₂, CH₂NHA, CH₂OH, CH₂OA, OH, NO₂, NH₂, NHA, NA₂, NH—CO—A, NH—CO—Ph, SA, SO-A, SO₂-A, SO₂-Ph, CN, OCF₃, CO-A, CO₂H, CO₂A, CO—NH₂, CO-NHA, CO-NA₂, SO₂NH₂, SO₂NHA, SO₂NA₂, CHO, or are phenyl, benzyl or cyclohexylmethyl, each of which is un-substituted or monosubstituted or polysubstituted by A, OH, OA, Hal, CN or CF₃, or are a heterocyclic radical which is monosubstituted or polysubstituted by A, OH, OA, Hal, CN or CF₃,
A is alkyl having 1, 2, 3, 4, 5 or 6 carbon atoms,
Hal is F, Cl, Br or I,
R⁵, R⁶, R⁷
and R⁸ are each, independently of one another, H, benzyl, allyl or another amino-protecting group, A, or phenyl, which is unsubstituted or monosubstituted or polysubstituted by A, OA, CN, Hal or CF₃, where R⁵ and R⁷, R⁵ and R⁶ and R⁷ and R⁸ may form 5-7-membered rings,
   and salts, solvates and stereoisomers thereof, including mixtures thereof in all ratios, and pharmaceutically usable derivatives thereof, in particular physiologically tolerated salts and solvates thereof.

The invention likewise relates to the use of the compounds of the formula I and salts and solvates thereof as NHE-3 inhibitors.

Other inhibitors of sodium/proton exchanger subtype 3 are described, for example in EP 0 825 178.

Quinazolinylguanidine derivatives have been described by V. I. Shvedov et al. in Pharm. Chem. J. (Engl. Transl.) 1980, 14, 532-538 or in Khim. Farm. Zh. 1980, 14, 38-43, and by S. C. Bell et al. in J. Med. Pharm. Chem. 1962, 5, 63-69.

The invention had the object of finding novel compounds having valuable properties, in particular those which can be used for the preparation of medicaments.

Surprisingly, it has been found that the compounds of the formula I and salts thereof are well tolerated and inhibit sodium/proton exchanger sub-type 3.

The compounds of the formula I can be employed as medicament active ingredients in human and veterinary medicine.

It is known that the Na⁺/H⁺+exchanger represents a family having at least 6 different isoforms (NHE-1 to NHE-6), all of which have already been cloned. While subtype NHE-1 is distributed ubiquitously in all tissues throughout the body, the other NHE subtypes are expressed selectively in specific organs, such as in the kidney or in the lumen wall and contraluminal wall of the small intestine. This distribution reflects the specific functions that the various isoforms serve, namely on the one hand regulation of the intracellular pH and cell volume by subtype NHE-1 and on the other hand Na⁺ absorption and resorption in the intestine and kidney by isoforms NHE-2 and NHE-3. Isoform NHE-4 has been found principally in the stomach. Expression of NHE-5 is restricted to the brain and neuronal tissue. NHE-6 is the isoform that forms the sodium/proton exchanger in the mitochondria.

Isoform NHE-3 is expressed in particular in the apical membrane of the proximal renal tubuli; an NHE-3 inhibitor therefore exerts, inter alia, a protective action on the kidneys.

The therapeutic use of a selective inhibitor for NHE-3 isoforms is manifold. NHE-3 inhibitors inhibit or reduce tissue damage and cell necrosis after pathophysiological hypoxic and ischaemic events which result in a reinforcement of the NHE activity, as is the case during renal ischaemia or during the removal, transport and reperfusion of a kidney during a kidney transplant.

The compounds of the formula I have a cytoprotective action in that they prevent the excessive absorption of sodium and water into the cells of organs undersupplied with oxygen.

The compounds of the formula I have a hypotensive action and are suitable as medicament active ingredients for the treatment of hypertonia.

They are furthermore suitable as diuretics.

The compounds of the formula I, alone or in combination with NHE inhibitors of other subtype specificity, have an antiischaemic action and can be used in the case of thromboses, atherosclerosis, vascular spasms, for the protection of organs, for example kidney and liver, before and during operations, and in the case of chronic or acute renal failure.

They can furthermore be used for the treatment of strokes, cerebral oedema, ischaemia of the nervous system, various forms of shock, for example allergic, cardiological, hypovolemic or bacterial shock, and for improving breathing drive in, for example, the following states: central sleep apnoea, cot death, postoperative hypoxia and other breathing disorders.

Through combination with a carboanhydrase inhibitor, breathing activity can be further improved.

The compounds of the formula I have an inhibiting effect on the proliferation of cells, for example fibroblast cell proliferation and the proliferation of the smooth vascular muscle cells, and can therefore be used for the treatment of diseases in which cell proliferation represents a primary or secondary cause.

The compounds of the formula I can be used against delayed complications of diabetes, cancer diseases, fibrotic diseases, endothelial dysfunction, organ hypertrophia and hyperplasia, in particular in prostate hyperplasia or prostate hypertrophia.

They are furthermore suitable as diagnostic agents for the determination and differentiation of certain forms of hypertonia, atherosclerosis, diabetes and proliferative diseases.

Since the compounds of the formula I also have an advantageous effect on the level of serum lipoproteins, they can be employed, alone or in combination with other medicaments, for the treatment of an increased blood fat level.

The invention relates to the use of compounds of the formula I according to claim 1 and physiologically acceptable salts and/or solvates thereof for the preparation of a medicament for the treatment of thromboses, ischaemic states of the heart, of the peripheral and central nervous system and of strokes, ischaemic states of peripheral organs and extremities and for the treatment of shock states.

The invention furthermore relates to the use of compounds of the formula I according to claim 1 and physiologically acceptable salts and/or solvates thereof for the preparation of a medicament for use in surgical operations and organ transplants and for the preservation and storage of transplants for surgical measures.

The invention also relates to the use of compounds of the formula I according to claim 1 and physiologically acceptable salts and/or solvates thereof for the preparation of a medicament for the treatment of diseases in which cell proliferation represents a primary or secondary cause, for the treatment or prophylaxis of disorders of fat metabolism or disturbed breathing drive.

The invention furthermore relates to the use of compounds of the formula I according to claim 1 and physiologically acceptable salts and/or solvates thereof for the preparation of a medicament for the treatment of renal ischaemia, ischaemic intestinal diseases or for the prophylaxis of acute or chronic renal diseases.

Methods for the identification of substances which inhibit sodium/proton exchanger subtype 3 are described, for example, in U.S. Pat. No. 5,871,919.

The compounds of the formula I are, in addition, suitable for the treatment of bacterial and parasitic diseases.

For all radicals in the compounds of the formula I which occur more than once, such as, for example, A, their meanings are independent of one another.

The term solvates of the compounds of the formula I is taken to mean adductions of water or other solvent molecules onto the compounds of the formula I which form owing to their mutual attractive force. Solvates are, for example, hemi-, mono- or dihydrates, alcohol addition compounds with, for example, methanol or ethanol, or ether addition compounds.

In the formulae above, A is alkyl, which is linear or branched and has 1, 2, 3, 4, 5 or 6 carbon atoms. A is preferably methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethyl-butyl, 1- or 2-ethylbutyl, 1-ethyl-1-methylpropyl, 1-ethyl-2-methylpropyl, 1,1,2- or 1,2,2-trimethylpropyl.

OA is preferably methoxy, ethoxy, propoxy, isopropoxy or butoxy.

Hal is preferably F, Cl or Br, in particular F or Cl.

The term “amino-protecting group” is known in general terms and relates to groups which are suitable for protecting (blocking) an amino group against chemical reactions, but can easily be removed after the desired chemical reaction has been carried out elsewhere in the molecule. Typical of such groups are, in particular, unsubstituted or substituted acyl, aryl, aralkoxymethyl or aralkyl groups. Since the amino-protecting groups are removed after the desired reaction (or reaction sequence), their nature and size are furthermore not crucial; however, preference is given to those having 1-20, in particular 1-8, carbon atoms. The term “acyl group” covers acyl groups derived from aliphatic, araliphatic, aromatic or heterocyclic carboxylic acids or sulfonic acids and in particular alkoxycarbonyl, aryloxycarbonyl and especially aralkoxycarbonyl groups. Examples of amino-protecting groups of this type are alkanoyl, such as acetyl, propionyl, butyryl; aralkanoyl, such as phenylacetyl; aroyl, such as benzoyl or toluyl; aryloxyalkanoyl, such as POA; alkoxycarbonyl, such as methoxycarbonyl, ethoxycarbonyl, 2,2,2-tri-chloroethoxycarbonyl, BOC (tert-butoxycarbonyl), 2-iodoethoxycarbonyl; alkenyloxycarbonyl, such as allyloxycarbonyl (Aloc), aralkyloxycarbonyl, such as CBZ (“carbobenzoxy”, synonymous with Z), 4-methoxybenzyloxy-carbonyl (MOZ), 4-nitrobenzyloxycarbonyl or 9-fluorenylmethoxycarbonyl (FMOC2-(phenylsulfonyl)ethoxycarbonyl; trimethylsilylethoxycarbonyl (Teoc), or arylsulfonyl, such as 4-methoxy-2,3,6-trimethylphenylsulfonyl (Mtr). The amino-protecting group is preferably formyl, acetyl, propionyl, butyryl, phenylacetyl, benzoyl, toluyl, POA, methoxycarbonyl, ethoxy-carbonyl, 2,2,2-trichloroethoxycarbonyl, BOC, 2-iodoethoxycarbonyl, CBZ (“carbobenzoxy”), 4-methoxybenzyloxycarbonyl, FMOC, Mtr or benzyl.

Above and below, Ph is preferably an unsubstituted phenyl radical, unless stated otherwise.

Het is preferably an aromatic and in particular saturated heterocyclic radical which is unsubstituted or substituted by A, OA and/or Hal. This heterocyclic radical can be monocyclic or polycyclic and is preferably monocyclic or bicyclic, but in particular monocyclic.

Above and below, the heterocyclic radical is preferably, for example, 2- or 3-furyl, 2- or 3-thienyl, 1-, 2- or 3-pyrrolyl, 1-, 2, 4- or 5-imidazolyl, 1-, 3-, 4- or 5-pyrazolyl, 2-, 4- or 5-oxazolyl, 3-, 4- or 5-isoxazolyl, 2-, 4- or 5-thia-zolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or 5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, 4- or 5-isoindolyl, 1-, 2-, 4- or 5-benzimidazolyl, 1-, 3-, 4-, 5-, 6- or 7-benzo-pyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, furthermore preferably 1,3-benzodioxol-5-yl, 1,4-benzodioxan-6-yl, 2,1,3-benzothiadiazol-4- or -5-yl or 2,1,3-benzoxadiazol-5-yl.

The heterocyclic radicals may also be partially or fully hydrogenated. The heterocyclic radical used can thus also be, for example, 2,3-dihydro-2-, -3-, -4- or -5-furyl, 2,5-dihydro-2-, -3-, -4- or 5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyra-zolyl, tetrahydro-1-, -3- or 4-pyrazolyl, 1,4-dihydro-1-, -2-, -3- or -4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxanyl, 1,3-dioxan-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, -4-, -5-, -6-, -7- or -8-quinolyl, 1,2,3,4-tetrahydro-1-, -2, -3-, -4-, -5-, -6-, -7- or -8-isoquinolyl, 2-, 3-, 5-, 6-, 7- or 8- 3,4-dihydro-2H-benzo-1,4-oxazinyl, furthermore preferably 2,3-methylenedioxyphenyl, 3,4-methylenedioxy-phenyl, 2,3-ethylenedioxyphenyl, 3,4-ethylenedioxyphenyl, 3,4-(difluoro-methylenedioxy)phenyl, 2,3-dihydrobenzofuran-5- or -6-yl, 2,3-(2-oxomethylenedioxy)phenyl or 3,4-dihydro-2H-1,5-benzodioxepin-6- or -7-yl, furthermore preferably 2,3-dihydrobenzofuranyl or 2,3-dihydro-2-oxo-furanyl.

The said heterocyclic radicals may additionally be substituted by A, OA and/or Hal.

The heterocyclic radical may furthermore preferably be selected from the following group:

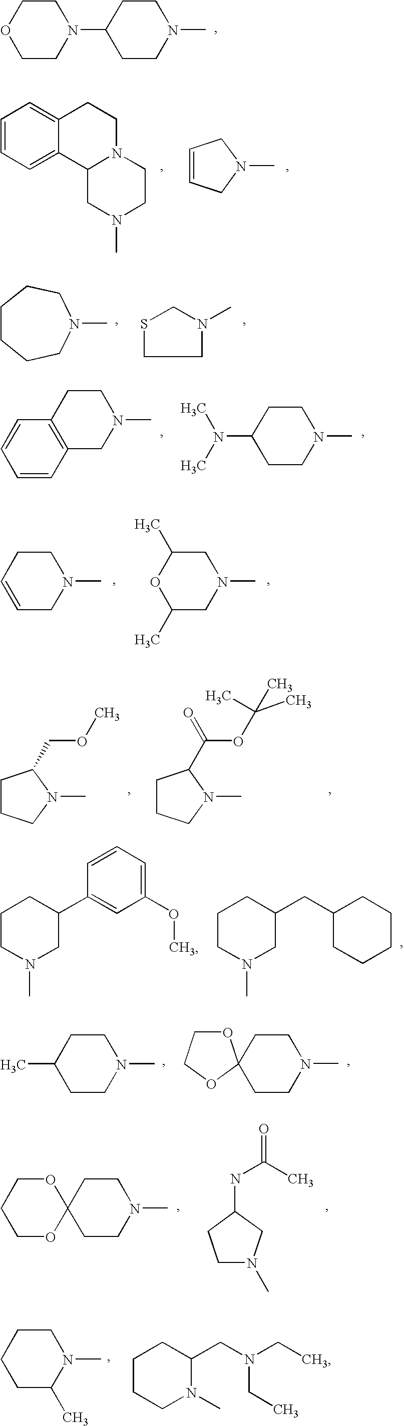

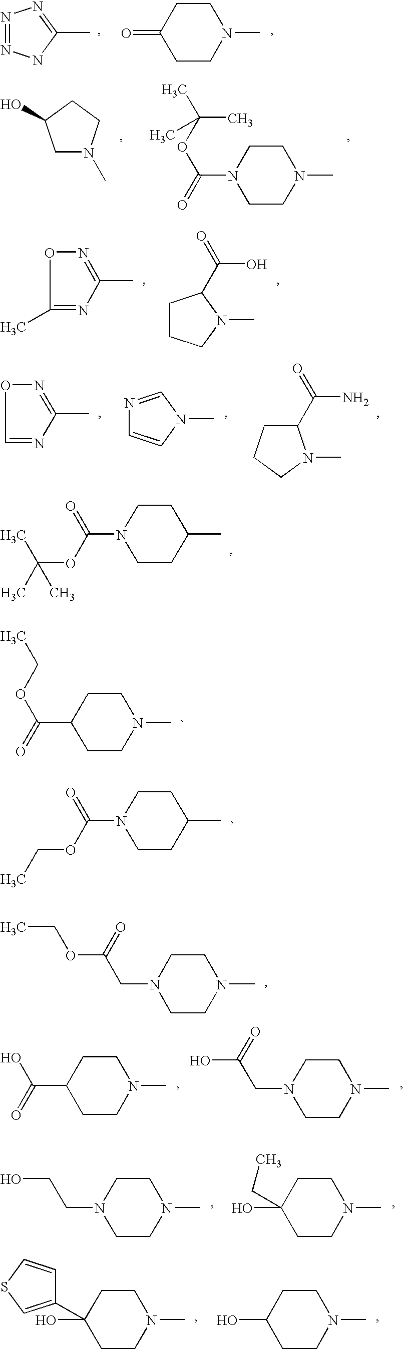

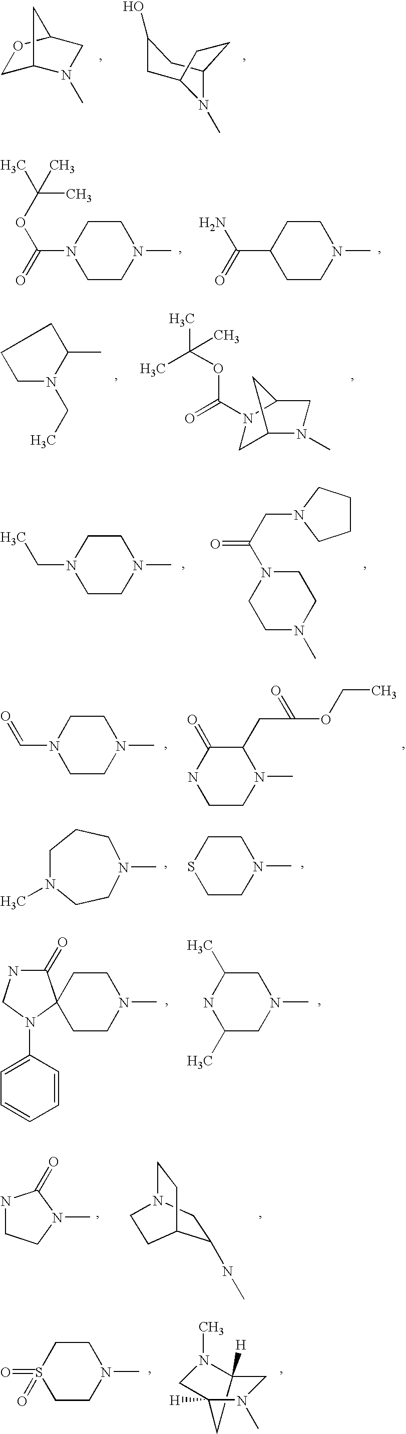

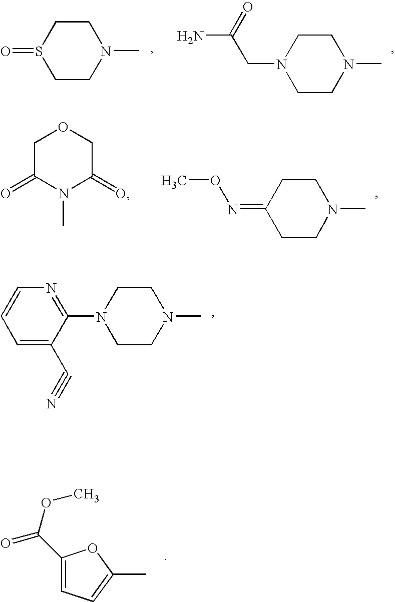

The heterocyclic radical is particularly preferably 2- or 3-furyl, 2- or 3-thienyl, 1-, 2- or 3-pyrrolyl, in particular 1-pyrrolyl, 1-, 2, 4- or 5-imidazolyi, 1-, 3-, 4- or 5-pyrazolyl, 2-, 4- or 5-oxazolyi, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or -5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2- or 3-pyrrolidinyl, 1-, 2-, 3- or 4-piperidinyl, 2-, 3- or 4-morpholinyl, 1,4-dioxanyl, 1,3-dioxan-2-, -4- or -5-yl, 1-, 2- or 3-piperazinyl.

The heterocyclic radical is furthermore preferably selected from the following group:

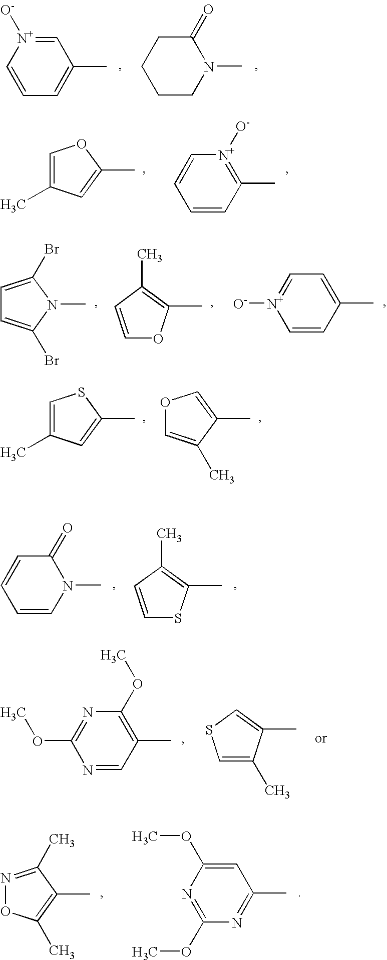

If a plurality of heterocyclic radicals occur in the compounds of the formula I, these may have identical or different meanings.

R¹, R², R³ and R⁴ are preferably, independently of one another, H, A, OA, Hal, CF₃, CH₂CONH₂, CH₂CO₂H, CH₂CO₂A, CH₂NH₂, CH₂NA₂, CH₂NHA, CH₂OH, CH₂OA, OH, NO₂, NH₂, NHA, NA₂, NH-CO-A, NH-CO-Ph, SA, SO-A, SO₂-A, SO₂-Ph, CN, OCF₃, CO-A, CO₂H, CO₂A, CO-NH₂, CO-NHA, CO-NA₂, SO₂NH₂, SO₂NHA or SO₂NA₂, in particular H, A, OA, Hal, CF₃, CH₂CONH₂, CH₂CO₂H, CH₂CO₂A, CH₂NH₂, OH, NO₂, NH₂, NHA, NA₂ or NH-CO-A.

R⁵ and R⁷ are particularly preferably simultaneously H, while R⁶ or R⁸ is H or A, but in particular H.

If at least one of the radicals R⁵, R⁶, R⁷ and R⁸ is H, the guanidino group Y may isomerise with respect to the double bond under generally known conditions. The formula I includes all isomers of this group.

If R⁵ and R⁷ together form a ring, Y preferably adopts one of the following structures: 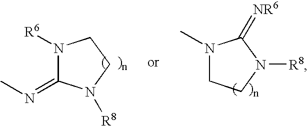
in which R⁶ and R⁸ are as defined above, and n is 1, 2 or 3, preferably 1 or 2.

If R⁷ and R⁸ together form a ring, Y preferably adopts one of the following structures: 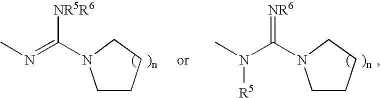
in which R⁵ and R⁶ are as defined above, and n is 1, 2 or 3, preferably 1 or 2.

If R⁵ and R⁶ together form a ring, Y preferably adopts one of the following structures: 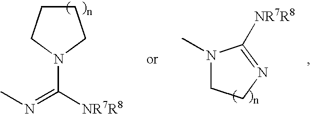
in which R⁷ and R⁸ are as defined above, and n is 1, 2 or 3, preferably 1 or 2.

The invention relates in particular to the compounds of the formula I in which at least one of the said radicals has one of the preferred meanings indicated above, and to the use thereof. Some preferred groups of compounds may be expressed by the following sub-formulae Ia to Ie, which conform to the formula I and in which the radicals not designated in greater detail have the meaning indicated in the formula I, but in which
in Ia R¹ is H, OH, OA, SA or Hal, in particular H;
in Ib R¹ is H, OH, OA, SA or Hal, in particular H,
R² is H, Hal, OH, A, NH₂, NO₂ or CN, in particular H, Cl, OH, CH₃ or NH₂;
in Ic R¹ is H, OH, OA, SA or Hal, in particular H, OH, OCH₃ or CH₃
R² is H, Hal, OH, A, NH₂, NO₂ or CN, in particular H, Cl, OH, CH₃ or NH₂.
Het is 2- or 3-furyl, 2- or 3-thienyl, 1-, 2- or 3-pyrrolyl, in particular 1-pyrrolyl, 1-, 2, 4- or 5-imidazolyl, 1-, 3-, 4- or 5-pyrazolyl, 2-, 4- or 5-oxazolyl, 3-, 4- or 5-isoxa-zolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, 4- or -5-yl, 1,2,4-triazol-1-, -3- or 5-yl, 1- or 5-tetrazolyl, 1,2,3-oxadiazol4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2- or 3-pyrroli-dinyl, 1-, 2-, 3- or 4-piperidinyl, 2-, 3- or 4-morpholinyl, 1,4-dioxanyl, 1,3-dioxan-2-, 4- or -5-yl, 1-, 2- or 3-piperazinyl,
R³ and R⁴ are H, A, OA or Hal, in particular H, Br, Cl, CH₃ or OCH₃;
in Id R¹ is H, OH, OA, SA or Hal, in particular H,
R² is H, Hal, OH, A, NH₂, NO₂ or CN, in particular H, Cl, OH, CH₃or NH₂.
Het is

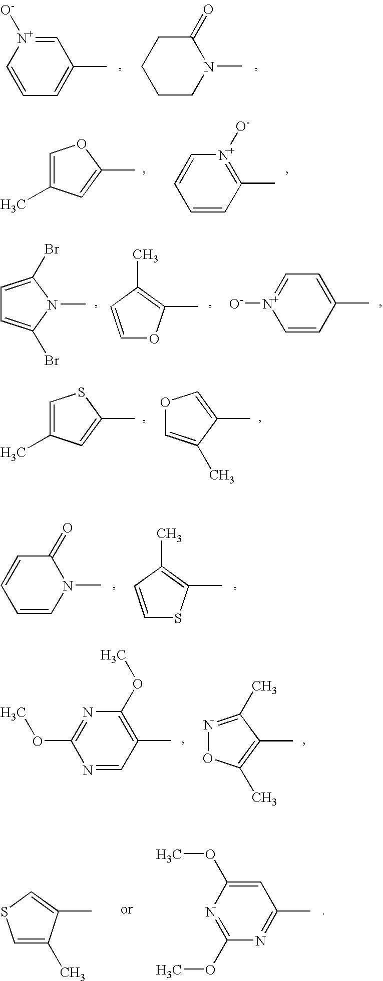

Preference is furthermore given to compounds of the formula I and salts and solvates thereof in which at least one of the radicals R¹, R², R³ and R⁴ has one of the following meanings:

OH, NO₂, NH₂, NHA, NA₂, NH-CO-A, NH-CO-Ph, SA, SO-A, SO₂-A, SO₂-Ph, CN, OCF₃, CO-A, CO₂H, CO₂A, CO-NH₂, CO-NHA, CO-NA₂, SO₂NH₂, SO₂NHA, SO₂NA₂, or phenyl which is unsubstituted or monosubstituted or polysubstituted by A, OA, Hal or CF₃.

Particular preference is furthermore given to the following compounds of the formulae IA, IB and IC: 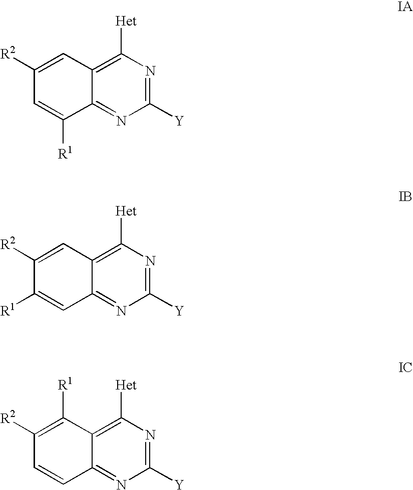
in which R¹, R², Het and Y are as defined-above, and R² is preferably Hal, in particular Cl.

Particular preference is given to compounds of the formulae ID 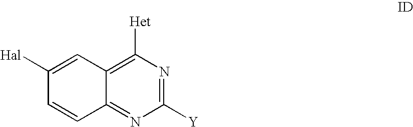
in which Hal is as defined above and is in particular Cl.

Compounds of the formula I whose radical R³ is methyl have particularly pronounced selectivity of binding to the NHE-3 receptor.

Compounds of the formula I whose radical R⁴ is NH₂ exhibit particularly good solubility in aqueous solutions.

Particular preference is given to the compounds of the formulae I1 to I14 and salts and solvates thereof:

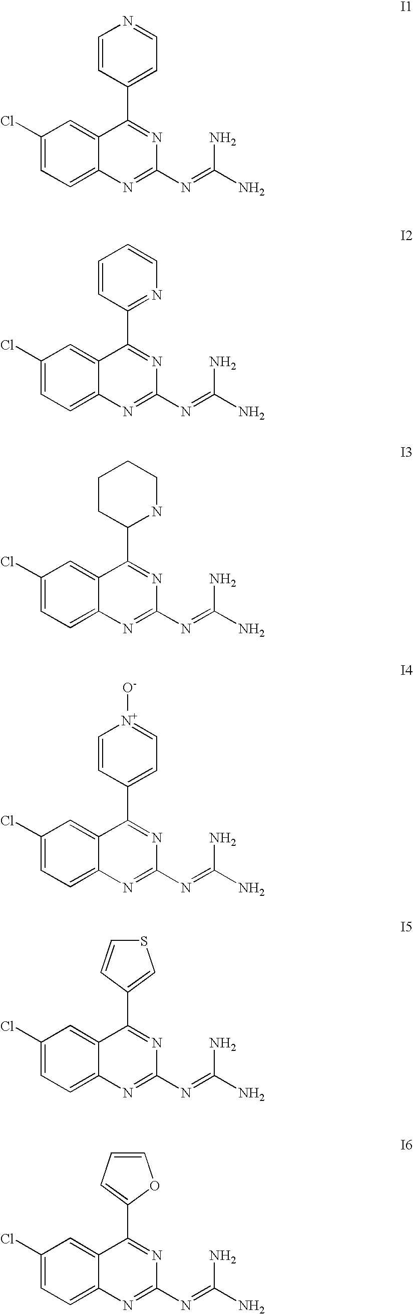

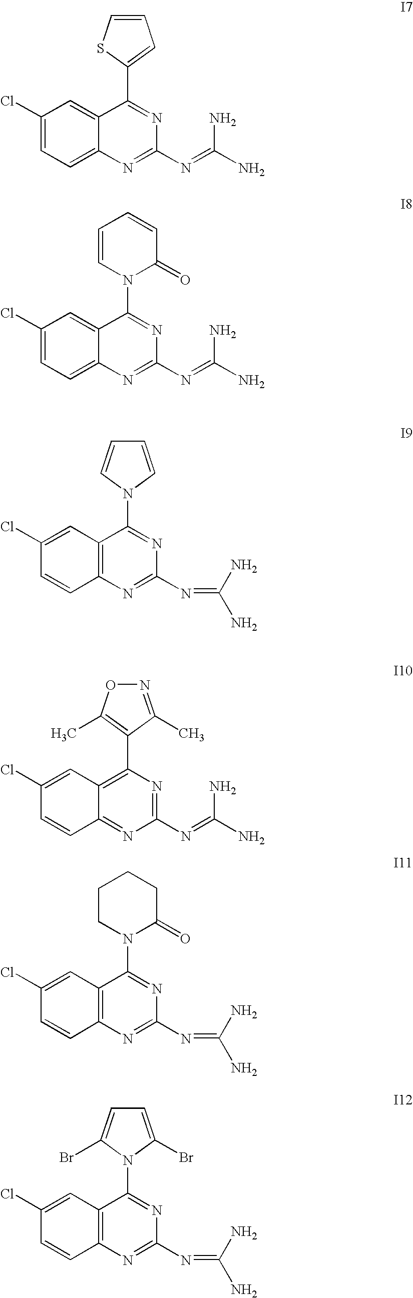

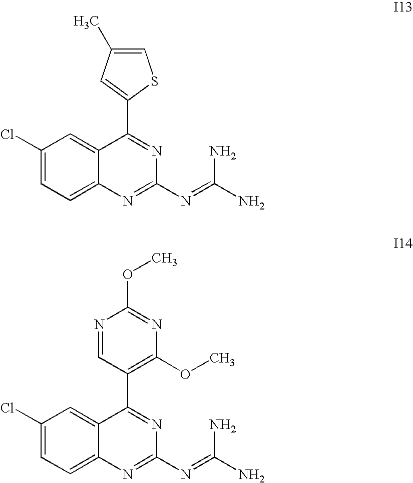

R² is preferably H, Cl, A, NH₂, NO₂, SCH₃, SOCH₃, SO₂CH₃, OCH₃, OH, CN, CF₃, OCF₃ or F, in particular H, Cl, F, Br, OH, CH₃, NO₂ or NH₂. R² is very particularly preferably Cl.

R³ is preferably H, Cl, OA, NH₂, NO₂, SCH₃, CN, C₂H₅, OCF₃ or C₆H₅, in particular H, OA or CH₃. R³ is very particularly preferably H or OCH₃.

R⁴ is preferably H, F, NH₂ or NO₂, in particular H or NH₂. R⁴ is very particularly preferably H or NH₂.

Y preferably adopts one of the following meanings:

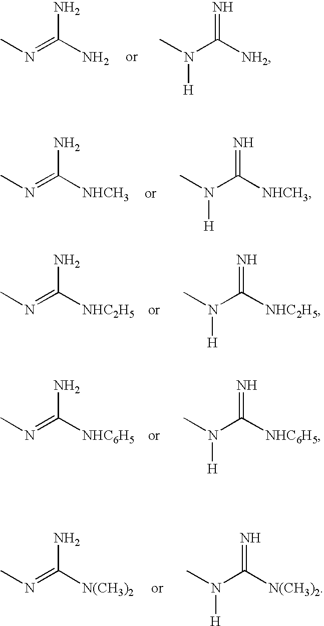

Y particularly preferably has one of the following meanings:

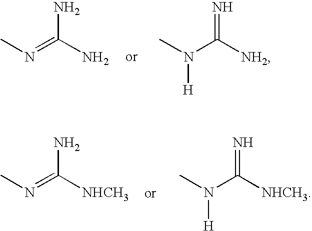

The hydrochlorides and p-toluenesulfonates of the compounds of the formulae I are very particularly preferred.

The compounds of the formula I may have one or more asymmetrically substituted carbon atoms and may accordingly occur as pure enantiomers or as a mixture of the enantiomers. Likewise, different diastereomers may arise in the presence of a plurality of asymmetrically substituted carbon atoms. The present invention likewise relates to the various diastereomers and enantiomers and mixtures thereof.

The compounds of the formula I and also the starting materials for the preparation thereof are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants which are known per se, but are not mentioned here in greater detail.

The starting materials can, if desired, also be formed in situ, so that they are not isolated from the reaction mixture, but instead are immediately converted further into the compounds of the formula I.

The compounds of the formula I are preferably prepared by reacting o-aminophenylheterocyclyl ketones or of the formula II 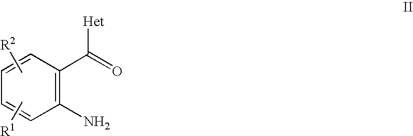
in which R¹, R² and Het are as defined in claim 1, with 1-cyanoguanidine or a correspondingly N-alkylated or N-arylated 1-cyanoguanidine of the formula NC-Y, in which Y is as defined above.

The reaction can be carried out in a solvent, preferably an inert solvent.

Examples of suitable solvents are hydrocarbons, such as hexane, petroleum ether, benzene, toluene or xylene; chlorinated hydrocarbons, such as trichloroethylene, 1,2-dichloroethane, tetrachloromethane, chloroform or dichloromethane; alcohols, such as methanol, ethanol, isopropanol, n-pro-panol, n-butanol or tert-butanol; ethers, such as diethyl ether, diisopropyl ether, tetrahydrofuran (THF) or dioxane; glycol ethers, such as ethylene glycol monomethyl or monoethyl ether, ethylene glycol dimethyl ether (diglyme); ketones, such as acetone or butanone; amides, such as acetamide, dimethylacetamide, N-methylpyrrolidone (NMP) or dimethyl-formamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitro-benzene; esters, such as ethyl acetate, or mixtures of the said solvents.

DMF, water or an alcohol is preferably used.

The reaction is very particularly preferably carried out without a solvent, i.e. in the melt, at temperatures between 100 and 200° C.

The presence of an acidic catalyst, such as AlCl₃, TiCl₄, p-toluenesulfonic acid, BF₃, acetic acid, sulfuric acid, oxalic acid, POCl₃ or phosphorus pentoxide, is advantageous.

A preferred variant comprises employing one of the reactants already as a salt, for example as the hydrochloride.

A further valuable method for the preparation of the compounds of the formula I comprises reacting, instead of a compound of the formula NC-Y, a compound of the formula III
HN═CX—Y III
in which

X is —S-alkyl, —S-aryl, —O-alkyl or —O-aryl, and alkyl is preferably as defined above for A, and aryl is preferably phenyl which is unsubstituted or monosubstituted or polysubstituted by A, OH, OA, Hal, CN or CF₃, with a compound of the formula II.

Finally, the compounds of the formula I can be prepared by reaction of compounds of the formula IV 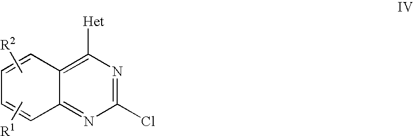
in which Het, R¹ and R² are as defined above, with a compound of the formula HY, in which Y is as defined above. HY is particularly preferably guanidine. This reaction is preferably carried out in the presence of a strong base, such as alkali metal alkoxide or strongly basic amines. The bases used are particularly preferably sodium methoxide, sodium ethoxide, potassium methoxide, potassium ethoxide, potassium tert-butoxide, DBN, DBU or DABCO.

The solvents used for the reaction of compounds of the formula IV with compounds of the formula HY are preferably DMSO, NMP or DMF.

The compounds of the formula IV can be obtained by preparation methods which are known per se.

The compounds of the formula IV are particularly preferably by reaction of the compounds of the formula V 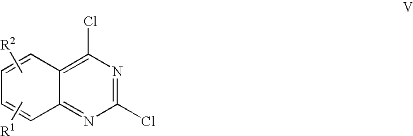
a) with heterocyclic boronic acids of the formula Het-B(OH)₂ in the presence of a palladium compound, such as, for example, bis(triphenylphosphine)-palladium(II) chloride in the form of a Suzuki coupling. Many variants of this reaction have already been disclosed in the literature (for example S. L. Buchwald and J. M. Fox, The Strem Chemiker 200, 18, 1).
b) with heterocyclic tributyltin compounds of the formula Het-Sn(n-C₂H₅)₃ in the form of a Stille coupling (for example J. K Stille Angew. Chem. Int. Ed. Engl. 1986, 25, 508).
   or
c) with heterocyclic nitrogen compounds having a free NH function, such as, for example, pyridonres or pyrrole, in the form of a nucleophilic displacement. The heterocyclic ring is then bonded via N. This reaction is preferably carried out in the presence of an acid scavenger, such as, for example, sodium hydride or potassium carbonate, and in the presence of a polar solvent, such as DMSO, NMP or DMF.

The present application likewise relates to the process for the preparation of the compounds of the formula V.

The present application likewise relates to the novel compounds of the formulae II and IV.

In some cases, it may be appropriate only to form the radicals R¹, R², R³ and R⁴ and other functional groups after the reaction of the compounds of the formula 11 with the compounds of the formula NC-Y or the compounds of the formula III, for example by removal of a protecting group, ether cleavage or hydrogenation of nitro groups to amino groups. Correspondingly, it may likewise be appropriate only to form the radicals R¹, R², R³ and R⁴ and other functional groups after the reaction of the compounds of the formula IV with the compounds of the formula HY by the above-mentioned measures.

A base of the formula I can be converted into the associated acid-addition salt using an acid, for example by reaction of equivalent amounts of the base and the acid in a preferably inert solvent, such as ethanol, followed by evaporation. Suitable acids for this reaction are, in particular, those which give physiologically acceptable salts. Thus, it is possible to use inorganic acids, for example sulfuric acid, nitric acid, hydrohalic acids, such as hydrochloric acid or hydrobromic acid, phosphoric acids, such as orthophosphoric acid, or sulfamic acid, furthermore organic acids, in particular aliphatic, alicyclic, araliphatic, aromatic or heterocyclic monobasic or polybasic carboxylic, sulfonic or sulfuric acids, for example formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, p-toluenesulfonic acid, naphthalenemono- and -disulfonic acids, and laurylsulfuric acid. Salts of compounds of the formula I with physiologically unacceptable acids, for example picrates, can be used for the isolation and/or purification of the compounds of the formula I and are likewise a subject-matter of the present invention.

The invention furthermore relates to the use of the compounds of the formula I as NHE-3 inhibitors and/or physiologically acceptable salts thereof for the preparation of pharmaceutical preparations, in particular by non-chemical methods. In this case, they can be converted into a suitable dosage form together with at least one solid, liquid and/or semi-liquid excipient or adjuvant, and, if desired, in combination with one or more further active ingredients.

The invention furthermore relates to pharmaceutical preparations comprising at least one NHE-3 inhibitor of the formula I and/or one of its physiologically acceptable salts and solvates.

These preparations can be used as medicaments in human or veterinary medicine. Suitable excipients are organic or inorganic substances which are suitable for enteral (for example oral), parenteral or topical administration and do not react with the novel compounds, for example water, vegetable oils, benzyl alcohols, alkylene glycols, polyethylene glycols, glycerol triacetate, gelatine, carbohydrates, such as lactose or starch, magnesium stearate, talc or Vaseline. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders, or transdermally in patches.

The novel compounds may also be lyophilised and the resultant lyophilisates used, for example, for the preparation of injection preparations. The preparations indicated may be sterilised and/or comprise adjuvants, such as lubricants, preservatives, stabilisers and/or wetting agents, emulsifiers, salts for modifying the osmotic pressure, buffer substances, colorants and flavours and/or a plurality of further active ingredients, for example one or more vitamins.

Suitable pharmaceutical preparations for administration in the form of aerosols or sprays are, for example, solutions, suspensions or emulsions of the active ingredient of the formula I in a pharmaceutically acceptable solvent.

The compounds of the formula I and physiologically acceptable salts and solvates thereof can be used for the treatment and/or prophylaxis of the diseases or disease states described above.

In general, the substances according to the invention are preferably administered in doses between about 0.1 and 100 mg, in particular between 1 and 10 mg, per dosage unit. The daily dose is preferably between about 0.001 and 10 mg/kg of body weight. However, the specific dose for each patient depends on a wide variety of factors, for example on the efficacy of the specific compound employed, on the age, body weight, general state of health, sex, on the diet, on the time and method of administration, on the excretion rate, medicament combination and severity of the particular disease to which the therapy applies. Oral administration is preferred.

### EXAMPLES

Above and below, all temperatures are indicated in ° C. In the following examples, “conventional work-up” means that water is added if necessary, the mixture is adjusted, if necessary, to a pH between 2 and 10, depending on the constitution of the end product, the mixture is extracted with ethyl acetate or dichloromethane, the phases are separated, the organic phase is dried over sodium sulfate and evaporated, and the product is purified by chromatography on silica gel and/or by crystallisation.

Mass spectrometry (MS): El (electron impact ionisation) M+FAB (fast atom bombardment) (M+H)⁺

### Example 1

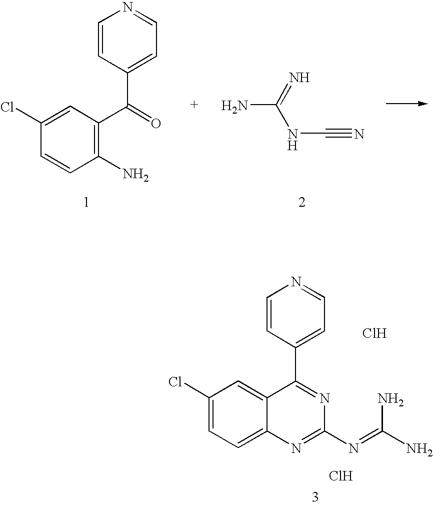

A mixture of 10.0 g of compound 1, 7.0 g of cyanoguanidine (2) and 22.0 g of p-toluenesulfonic acid is heated at 160° C. for one hour. The reaction mixture is heated with 80 ml and methanol, rendered alkaline using a 1 N aqueous solution of sodium hydroxide and filtered. The residue is subjected to conventional work-up and treated with a solution of hydrogen chloride gas in isopropanol, giving, after filtration, the product 3 (m.p.: 345° C.).

### Example 2

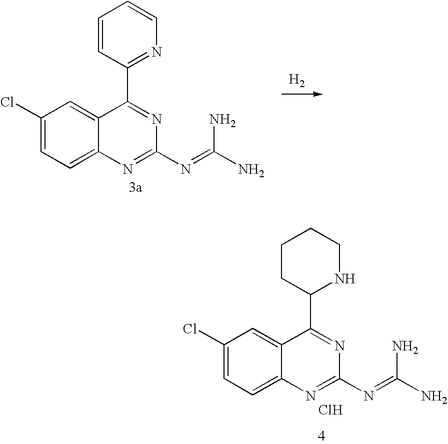

A solution of 200 mg of compound 3a (obtainable by liberation of the base from the corresponding hydrochloride) in 40 ml of methanol is hydrogenated at atmospheric pressure in the presence of PtVC (5%). The solvent is removed, and the residue is subjected to conventional work-up, giving, after addition of methanolic hydrochloric acid solution and filtration, the product 4.

### Example 3

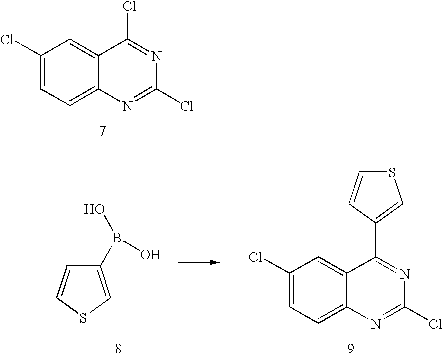

A mixture of 1.35 g of compound 7 (obtainable by the method of Okabe et al., Tetrahedron 1995, 51, 1861-1866), 0.75 g of the boronic acid (8), 309 mg of sodium hydroxide and 116 mg of tetrakis(triphenylphosphine)-palladium(0) in 19 ml of diethylene glycol dimethyl ether is heated at 130° C. for six hours. Water is subsequently added to the reaction mixture, which is worked up, giving the product 9 (m.p.: 174-176° C.).

### Example 4

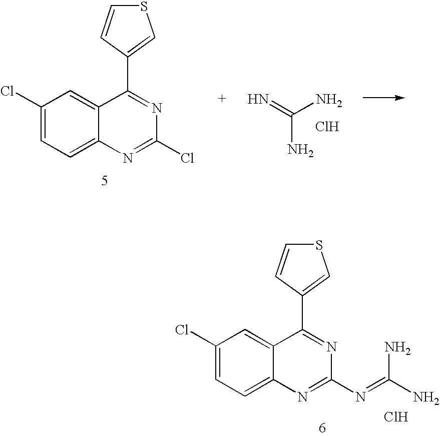

A mixture of 1.10 g of compound 5, 1.82 g of guanidinium chloride and 2.89 g of 1,8-diazabicyclo[5.4.0]undec-7-ene in 10.0 ml of 1-methyl-2-pyrrolidone is stirred overnight at room temperature. The reaction mixture is subjected to conventional work-up, giving, after addition of methanolic hydrochloric acid solution, the product 6 (m.p.: 294-297° C.).

### Example 5

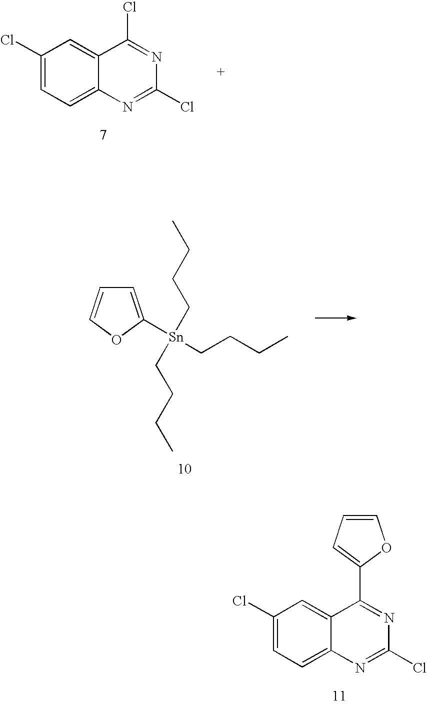

A mixture of 0.50 g of compound 7, 0.765 g of 2-(tributylstannyl)furan (10) and 0.150 g of bis(triphenylphosphine)palladium(II) chloride in 25 ml of dioxane is refluxed for two hours. The solvent is removed, and the residue is subjected to conventional work-up, giving compound 11.

### Example 6

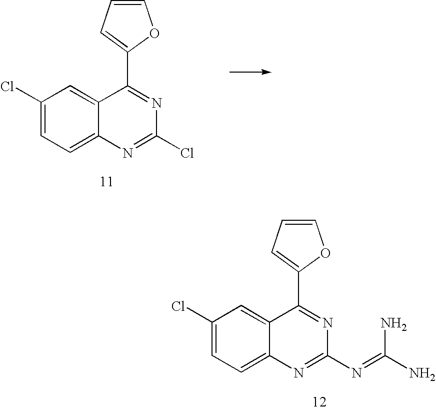

0.901 g of guanidinium chloride is stirred at room temperature for 30 minutes with 1.75 ml of a 30 per cent solution of sodium methoxide in methanol. The solvent is subsequently removed, and a solution of 0.25 g of compound 11 in 10 ml of dimethylformamide is added to the residue. The mixture is stirred at room temperature for two hours and subsequently subjected to conventional work-up, giving compound 12 (m.p.: 209-212° C.).

### Example 7

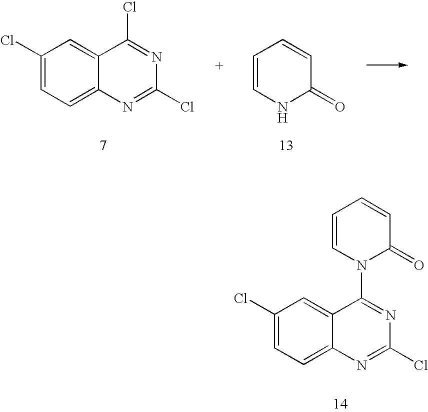

1.05 g of compound 7, 0.55 g of 13 and 2.0 g of potassium carbonate are stirred overnight at room temperature in 15 ml of dimethylformamide. The reaction mixture is subsequently diluted with water and filtered. Conventional work-up of the residue gives the product 14.

### Example 8

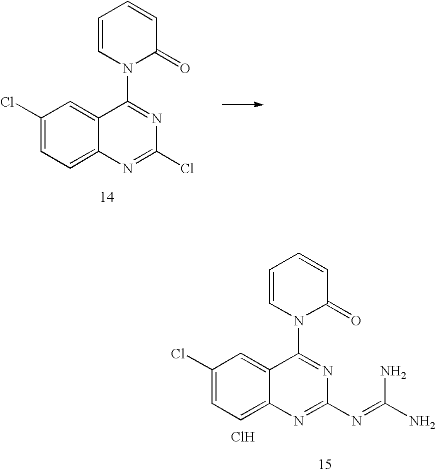

250 mg of compound 14 are dissolved in 3 ml of dimethyl sulfoxide, and 100 mg of DABCO are added. A stoichiometric amount of guanidine base (liberated from guanidinium chloride by sodium methoxide) in dimethyl sulfoxide is subsequently added, and the mixture is stirred at room temperature for 30 minutes. After addition of water, the mixture is filtered, and the residue is subjected to conventional work-up and, after addition of a solution of HCl in isopropanol and filtration, converted into the product 15 (m.p.: 285 degrees).

### Example 9

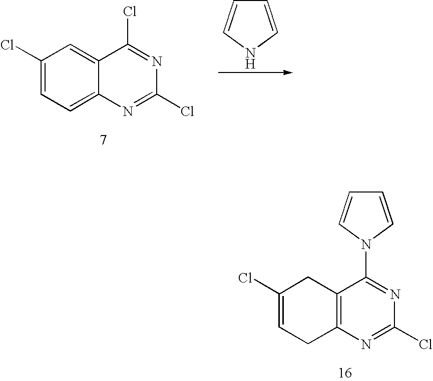

340 mg of sodium in white oil are added under a nitrogen atmosphere to a solution of 0.70 ml of pyrrole in 10 ml of dimethyl sulfoxide, and the mixture is stirred for 30 minutes. The resultant solution is added dropwise with cooling to a solution of 2.33 g of compound 7 in 10 ml of dimethyl sulfoxide, and the mixture is stirred for a further two hours. Water is subsequently added to the reaction mixture, which is subjected to conventional work-up, giving the product 16.

### Example 10

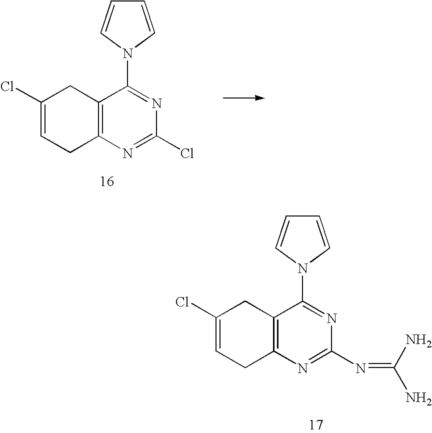

225 mg of DABCO are added to a solution of 528 mg of compound 16 in 5 ml of dimethyl sulfoxide, and the mixture is stirred for 30 minutes. 0.10 ml of guanidine base is subsequently added, and the mixture is stirred for a further 30 minutes. After addition of water, the mixture is subjected to conventional work-up, giving the product 17 (m.p.: 153° C.).

### Example 11

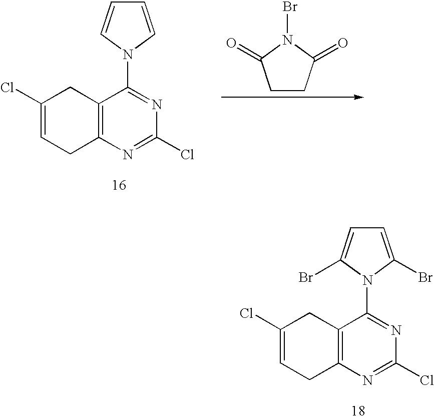

530 mg of compound 16 are dissolved in 10 ml of tetrahydrofuran, 340 mg of NBS are added, and the mixture is stirred at room temperature for two hours. After addition of a further 250 mg of NBS, the reaction mixture is stirred for two hours, diluted with water and subjected to conventional work-up, giving the product 18.

The corresponding guanidine compound is obtained from compound 18 analogously to Example 10.

The following compounds were obtained as NHE-3 inhibitors in the form of preferred acid-addition salts thereof analogously to the above-mentioned processes using the corresponding precursors:

pTsOH below denotes p-toluenesulfonic acid.

### Examples 12-29

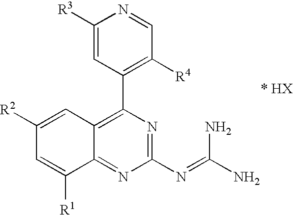

| | R¹ | R² | R³ | R⁴ | HX |
|---|---|---|---|---|---|
| (12) | H | OCH₃ | H | H | pTsOH |
| (13) | H | OCH₃ | H | H | HCl |
| (14) | H | Cl | H | Methyl | HCl |
| (15) | H | Cl | H | Ethyl | HCl |
| (16) | H | Cl | H | CN | pTsOH |
| (17) | H | Cl | H | NO₂ | pTsOH |
| (18) | H | Cl | H | NH₂ | pTsOH |
| (19) | H | Cl | H | CF₃ | HCl |
| (20) | H | Cl | H | OCH₃ | pTsOH |
| (21) | H | Cl | H | SO₂CH₃ | HCl |
| (22) | H | Cl | Methyl | H | HCl |
| (23) | H | Cl | Ethyl | H | HCl |
| (24) | H | Cl | CN | H | HCl |
| (25) | H | Cl | NO₂ | H | HCl |
| (26) | H | Cl | NH₂ | H | HCl |
| (27) | H | Cl | CF₃ | H | HCl |
| (28) | H | Cl | OCH₃ | H | HCl |
| (29) | H | Cl | SO_{2CH}₃ | H | HCl |

### Examples 30-47

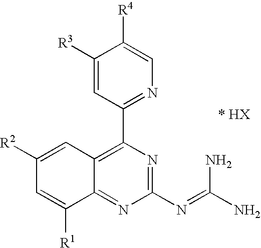

| | R¹ | R² | R³ | R⁴ | HX | |
|---|---|---|---|---|---|---|
| (30) | H | Cl | H | H | pTsOH | (m.p.: 208° C.) |
| (31) | H | Cl | H | H | HCl | |
| (32) | H | Cl | H | Methyl | HCl | |
| (33) | H | Cl | H | Ethyl | HCl | |
| (34) | H | Cl | H | CN | pTsOH | |
| (35) | H | Cl | H | NO₂ | pTsOH | |
| (36) | H | Cl | H | NH₂ | pTsOH | |
| (37) | H | Cl | H | CF₃ | HCl | |
| (38) | H | Cl | H | OCH₃ | pTsOH | |
| (39) | H | Cl | H | SO₂CH₃ | HCl | |
| (40) | H | Cl | Methyl | H | HCl | |
| (41) | H | Cl | Ethyl | H | HCl | |
| (42) | H | Cl | CN | H | HCl | |
| (43) | H | Cl | NO₂ | H | HCl | |
| (44) | H | Cl | NH₂ | H | HCl | |
| (45) | H | Cl | CF₃ | H | HCl | |
| (46) | H | Cl | OCH₃ | H | HCl | |
| (47) | H | Cl | SO₂CH₃ | H | HCl | |

### Examples 48-65

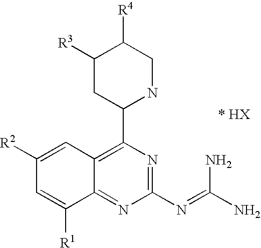

| | R¹ | R² | R³ | R⁴ | HX |
|---|---|---|---|---|---|
| (48) | H | Cl | H | H | pTsOH |
| (49) | H | OCH₃ | H | H | HCl |
| (50) | H | Cl | H | Methyl | HCl |
| (51) | H | Cl | H | Ethyl | HCl |
| (52) | H | Cl | H | CN | pTsOH |
| (53) | H | Cl | H | NO₂ | pTsOH |
| (54) | H | Cl | H | NH₂ | pTsOH |
| (55) | H | Cl | H | CF₃ | HCl |
| (56) | H | Cl | H | OCH₃ | pTsOH |
| (57) | H | Cl | H | SO₂CH₃ | HCl |
| (58) | H | Cl | Methyl | H | HCl |
| (59) | H | CI | Ethyl | H | HCl |
| (60) | H | Cl | CN | H | HCl |
| (61) | H | Cl | NO₂ | H | HCl |
| (62) | H | Cl | NH₂ | H | HCl |
| (63) | H | Cl | CF₃ | H | HCl |
| (64) | H | Cl | OCH₃ | H | HCl |
| (65) | H | Cl | SO₂CH₃ | H | HCl |

### Examples 66-83

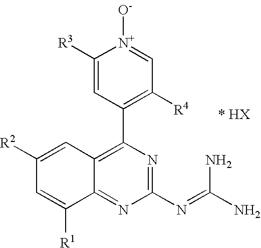

| | R¹ | R² | | R³ | R⁴ | HX |
|---|---|---|---|---|---|---|
| (66) | H | Cl | H | H | pTsOH | (m.p.: 305° C., |
| | | | | | | decomp.) |
| (67) | H | Cl | H | H | HCl | |
| (68) | H | Cl | H | Methyl | HCl | |
| (69) | H | Cl | H | Ethyl | HCl | |
| (70) | H | Cl | H | CN | pTsOH | |
| (71) | H | Cl | H | NO₂ | pTsOH | |
| (72) | H | Cl | H | NH₂ | pTsOH | |
| (73) | H | Cl | H | CF₃ | HCl | |
| (74) | H | Cl | H | OCH₃ | pTsOH | |
| (75) | H | Cl | H | SO₂CH₃ | HCl | |
| (76) | H | Cl | Methyl | H | HCl | |
| (77) | H | Cl | Ethyl | H | HCl | |
| (78) | H | Cl | CN | H | HCl | |
| (79) | H | Cl | NO₂ | H | HCl | |
| (80) | H | Cl | NH₂ | H | HCl | |
| (81) | H | Cl | CF₃ | H | HCl | |
| (82) | H | Cl | OCH₃ | H | HCl | |
| (83) | H | Cl | SO₂CH₃ | H | HCl | |

### Examples 84-101

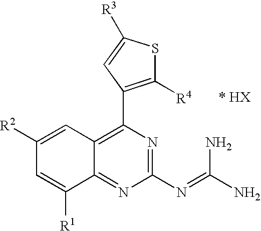

| | R¹ | R² | R³ | R⁴ | HX |
|---|---|---|---|---|---|
| (84) | H | Cl | H | H | pTsOH |
| (85) | H | OCH₃ | H | H | HCl |
| (86) | H | Cl | H | Methyl | HCl |
| (87) | H | Cl | H | Ethyl | HCl |
| (88) | H | Cl | H | CN | pTsOH |
| (89) | H | Cl | H | NO₂ | pTsOH |
| (90) | H | Cl | H | NH₂ | pTsOH |
| (91) | H | Cl | H | CF₃ | HCl |
| (92) | H | Cl | H | OCH₃ | pTsOH |
| (93) | H | Cl | H | SO₂CH₃ | HCl |
| (94) | H | Cl | Methyl | H | HCl |
| (95) | H | Cl | Ethyl | H | HCl |
| (96) | H | Cl | CN | H | HCl |
| (97) | H | Cl | NO₂ | H | HCl |
| (98) | H | Cl | NH₂ | H | HCl |
| (99) | H | Cl | CF₃ | H | HCl |
| (100) | H | Cl | OCH₃ | H | HCl |
| (101) | H | Cl | SO₂CH₃ | H | HCl |

### Examples 102-119

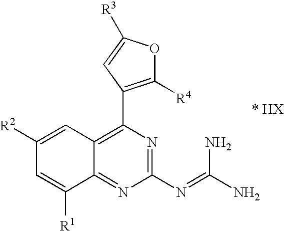

| | R¹ | R² | R³ | R⁴ | HX |
|---|---|---|---|---|---|
| (102) | H | Cl | H | H | pTsOH |
| (103) | H | Cl | H | H | HCl |
| (104) | H | Cl | H | Methyl | HCl |
| (105) | H | Cl | H | Ethyl | HCl |
| (106) | H | Cl | H | CN | pTsOH |
| (107) | H | Cl | H | NO₂ | pTsOH |
| (108) | H | Cl | H | NH₂ | pTsOH |
| (109) | H | Cl | H | CF₃ | HCl |
| (110) | H | Cl | H | OCH₃ | pTsOH |
| (111) | H | Cl | H | SO₂CH₃ | HCl |
| (112) | H | Cl | Methyl | H | HCl |
| (113) | H | Cl | Ethyl | H | HCl |
| (114) | H | Cl | CN | H | HCl |
| (115) | H | Cl | NO₂ | H | HCl |
| (116) | H | Cl | NH₂ | H | HCl |
| (117) | H | Cl | CF₃ | H | HCl |
| (118) | H | Cl | OCH₃ | H | HCl |
| (119) | H | Cl | SO₂CH₃ | H | HCl |

### Examples 120-137

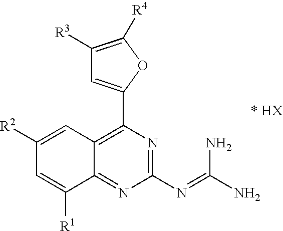

| | R¹ | R² | R³ | R⁴ | HX |
|---|---|---|---|---|---|
| (120) | H | Cl | H | H | pTsOH |
| (121) | H | Cl | H | H | HCl |
| (122) | H | Cl | H | Methyl | HCl |
| (123) | H | Cl | H | Ethyl | HCl |
| (124) | H | Cl | H | CN | pTsOH |
| (125) | H | Cl | H | NO₂ | pTsOH |
| (126) | H | Cl | H | NH₂ | pTsOH |
| (127) | H | CI | H | CF₃ | HCl |
| (128) | H | Cl | H | OCH₃ | pTsOH |
| (129) | H | Cl | H | SO₂CH₃ | HCl |
| (130) | H | Cl | Methyl | H | HCl |
| (131) | H | Cl | Ethyl | H | HCl |
| (132) | H | Cl | CN | H | HCl |
| (133) | H | Cl | NO₂ | H | HCl |
| (134) | H | Cl | NH₂ | H | HCl |
| (135) | H | Cl | CF₃ | H | HCl |
| (136) | H | Cl | OCH₃ | H | HCl |
| (137) | H | Cl | SO₂CH₃ | H | HCl |

### Examples 138-155

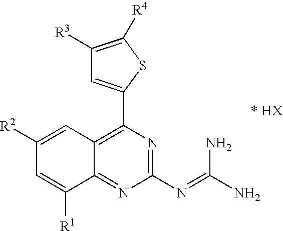

| | R¹ | R² | R³ | R⁴ | HX | |
|---|---|---|---|---|---|---|
| (138) | H | Cl | H | H | pTsOH | |
| (139) | H | Cl | H | H | HCl | (m.p.: 297° C.) |
| (140) | H | Cl | H | Methyl | HCl | |
| (141) | H | Cl | H | Ethyl | HCl | |
| (142) | H | Cl | H | CN | pTsOH | |
| (143) | H | Cl | H | NO₂ | pTsOH | |
| (144) | H | Cl | H | NH₂ | pTsOH | |
| (145) | H | Cl | H | CF₃ | HCl | |
| (146) | H | Cl | H | OCH₃ | pTsOH | |
| (147) | H | Cl | H | SO₂CH₃ | HCl | |
| (148) | H | Cl | Methyl | H | HCl | (m.p.: 296-299° C.) |
| (149) | H | Cl | Ethyl | H | HCl | |
| (150) | H | Cl | CN | H | HCl | |
| (151) | H | Cl | NO₂ | H | HCl | |
| (152) | H | Cl | NH₂ | H | HCl | |
| (153) | H | Cl | CF₃ | H | HCl | |
| (154) | H | Cl | OCH₃ | H | HCl | |
| (155) | H | Cl | SO₂CH₃ | H | HCl | |

### Examples 156-173

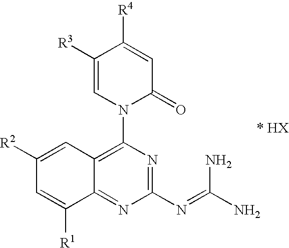

| | R¹ | R² | R³ | R⁴ | HX |
|---|---|---|---|---|---|
| (156) | H | Cl | H | H | pTsOH |
| (157) | H | OCH₃ | H | H | HCl |
| (158) | H | Cl | H | Methyl | HCl |
| (159) | H | Cl | H | Ethyl | HCl |
| (160) | H | Cl | H | CN | pTsOH |
| (161) | H | Cl | H | NO₂ | pTsOH |
| (162) | H | Cl | H | NH₂ | pTsOH |
| (163) | H | Cl | H | CF₃ | HCl |
| (164) | H | Cl | H | OCH₃ | pTsOH |
| (165) | H | Cl | H | SO₂CH₃ | HCl |
| (166) | H | Cl | Methyl | H | HCl |
| (167) | H | Cl | Ethyl | H | HCl |
| (168) | H | Cl | CN | H | HCl |
| (169) | H | Cl | NO₂ | H | HCl |
| (170) | H | Cl | NH₂ | H | HCl |
| (171) | H | Cl | CF₃ | H | HCl |
| (172) | H | Cl | OCH₃ | H | HCl |
| (173) | H | Cl | SO₂CH₃ | H | HCl |

### Examples 174-191

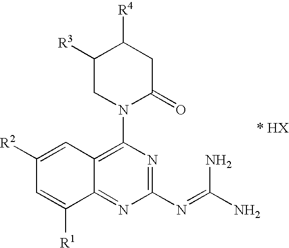

| | R¹ | R² | R³ | R⁴ | HX | |
|---|---|---|---|---|---|---|
| (174) | H | Cl | H | H | pTsOH | |
| (175) | H | Cl | H | H | HCl | (m.p.: 245° C.) |
| (176) | H | Cl | H | Methyl | HCl | |
| (177) | H | Cl | H | Ethyl | HCl | |
| (178) | H | Cl | H | CN | pTsOH | |
| (179) | H | Cl | H | NO₂ | pTsOH | |
| (180) | H | Cl | H | NH₂ | pTsOH | |
| (181) | H | Cl | H | CF₃ | HCl | |
| (182) | H | Cl | H | OCH₃ | pTsOH | |
| (183) | H | Cl | H | SO₂CH₃ | HCl | |
| (184) | H | Cl | Methyl | H | HCl | |
| (185) | H | Cl | Ethyl | H | HCl | |
| (186) | H | Cl | CN | H | HCl | |
| (187) | H | Cl | NO₂ | H | HCl | |
| (188) | H | Cl | NH₂ | H | HCl | |
| (189) | H | Cl | CF₃ | H | HCl | |
| (190) | H | Cl | OCH₃ | H | HCl | |
| (191) | H | Cl | SO₂CH3 | H | HCl | |

### Examples 192-212

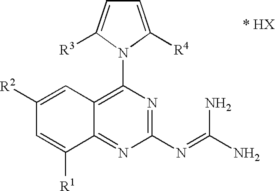

| | R¹ | R² | R³ | R⁴ | HX | |
|---|---|---|---|---|---|---|
| (192) | H | Cl | H | H | pTsOH | |
| (193) | H | OCH₃ | H | H | HCl | |
| (194) | H | Cl | Br | H | HCl | |
| (195) | H | Cl | H | Br | HCl | |
| (196) | H | Cl | Br | Br | HCl | (m.p.: 302° C.) |
| (197) | H | Cl | H | Methyl | HCl | |
| (198) | H | Cl | H | Ethyl | HCl | |
| (199) | H | Cl | H | CN | pTsOH | |
| (200) | H | Cl | H | NO₂ | pTsOH | |
| (201) | H | Cl | H | NH₂ | pTsOH | |
| (202) | H | Cl | H | CF₃ | HCI | |
| (203) | H | Cl | H | OCH₃ | pTsOH | |
| (204) | H | Cl | H | SO₂CH₃ | HCl | |
| (205) | H | Cl | Methyl | H | HCl | |
| (206) | H | Cl | Ethyl | H | HCl | |
| (207) | H | Cl | CN | H | HCl | |
| (208) | H | Cl | NO₂ | H | HCl | |
| (209) | H | Cl | NH₂ | H | HCl | |
| (210) | H | Cl | CF₃ | H | HCl | |
| (211) | H | Cl | OCH₃ | H | HCl | |
| (212) | H | Cl | SO₂CH₃ | H | HCl | |

### Examples 213-232

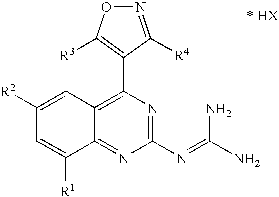

| | R¹ | R² | R³ | R⁴ | HX | |
|---|---|---|---|---|---|---|
| (213) | H | Cl | H | H | pTsOH | |
| (214) | H | Cl | H | H | HCl | |
| (215) | H | Cl | Methyl | Methyl | HCl | (m.p.: 317-320° C.) |
| (216) | H | Cl | Ethyl | Ethyl | HCl | |
| (217) | H | Cl | H | Methyl | HCl | |
| (218) | H | Cl | H | Ethyl | HCl | |
| (219) | H | Cl | H | ON | pTsOH | |
| (220) | H | Cl | H | NO₂ | pTsOH | |
| (221) | H | Cl | H | NH₂ | pTsOH | |
| (222) | H | Cl | H | CF₃ | HCl | |
| (223) | H | CI | H | OCH₃ | pTsOH | |
| (224) | H | Cl | H | SO₂CH₃ | HCl | |
| (225) | H | Cl | Methyl | H | HCl | |
| (226) | H | Cl | Ethyl | H | HCl | |
| (227) | H | Cl | ON | H | HCl | |
| (228) | H | Cl | NO₂ | H | HCl | |
| (229) | H | Cl | NH₂ | H | HCl | |
| (230) | H | Cl | CF₃ | H | HCl | |
| (231) | H | Cl | OCH₃ | H | HCl | |
| (232) | H | Cl | SO₂CH₃ | H | HCl | |

### Examples 233-252

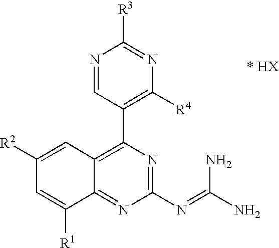

| | R¹ | R² | R³ | R⁴ | HX | |
|---|---|---|---|---|---|---|
| (233) | H | Cl | H | H | pTsOH | |
| (234) | H | Cl | H | H | HOl | |
| (235) | H | Cl | OCH₃ | OCH₃ | HCl | (m.p.: 270-275° C.) |
| (236) | H | Cl | OCF₃ | OCF₃ | HCl | |
| (237) | H | Cl | H | Methyl | HCl | |
| (238) | H | Cl | H | Ethyl | HCl | |
| (239) | H | Cl | H | ON | pTsOH | |
| (240) | H | Cl | H | NO₂ | pTsOH | |
| (241) | H | Cl | H | NH₂ | pTsOH | |
| (242) | H | Cl | H | CF₃ | HCl | |
| (243) | H | Cl | H | OCH₃ | pTsOH | |
| (244) | H | Cl | H | SO₂CH₃ | HCl | |
| (245) | H | Cl | Methyl | H | HCl | |
| (246) | H | Cl | Ethyl | H | HCl | |
| (247) | H | Cl | ON | H | HCl | |
| (248) | H | Cl | NO₂ | H | HCl | |
| (249) | H | Cl | NH₂ | H | HCl | |
| (250) | H | Cl | CF₃ | H | HCl | |
| (251) | H | Cl | OCH₃ | H | HCl | |
| (252) | H | Cl | SO₂CH₃ | H | HCl | |

Pharmacological Tests

The method used for the characterisation of the compounds of the formula I as NHE-3 inhibitors is described below.

The compounds of the formula I are characterised with respect to their selectivity for the NHE-1 to NHE-3 isoforms. The three isoforms are expressed in stable form in mouse fibroblast cell lines. The inhibitory action of the compounds is assessed by determination of the ElPA-sensitive up-take of ²²Na⁺ into the cells after intracellular acidosis.

Material and Methods

LAP1 Cell Lines which Express the Different NHE Isoforms

The LAP1 cell lines which express the NHE-1, -2 and -3 isoforms (a mouse fibroblast cell line) were obtained from Prof. J. Pouysségur (Nice, France). The transfection is carried out by the method of Franchi et al. (1986). The cells are cultivated in Dulbeccos modified eagle medium (DMEM) with 10% of deactivated foetal calf serum (FCS). For selection of the NHE-expressing cells, the so-called “acid killing method” of Sardet et al. (1989) is used. The cells are firstly incubated for 30 minutes in an NH₄Cl-containing bicarbonate- and sodium-free buffer. The extracellular NH₄Cl is then removed by washing with a bicarbonate-, NH₄Cl- and sodium-free buffer. The cells are subsequently incubated in a bicarbonate-free, NaCl-containing buffer. Only those cells which functionally express NHE are able to survive in the intracellular acidification to which they are subjected.

Characterisation of NHE Inhibitors with Respect to their Isoform Selectivity

With the above-mentioned mouse fibroblast cell lines which express the NHE-1, NHE-2 and NHE-3 isoforms, compounds are tested for selectivity with respect to the isoforms by the procedure described by Counillon et al. (1993) and Scholz et al. (1995). The cells are acidified intracellularly by the NH₄Cl prepulse method and subsequently by incubation in a bicarbonate-free ²²Na⁺-containing buffer. Owing to the intracellular acidification, NHE is activated, and sodium is taken up into the cells. The effect of the test compound is expressed as inhibition of EIPA (ethylisopropylamiloride)-sensitive ²²Na⁺ uptake.

The cells which expressed NHE-1, NHE-2 and NHE-3 are sown out in a density of 5-7.5×10⁴ cells/well in 24-well microtitre plates and cultured to confluence for from 24 to 48 hours. The medium is removed by suction, and the cells are incubated for 60 minutes at 37° C. in NH₄Cl. buffer (50 mM NH₄Cl, 70 mM choline chloride, 15 mM MOPS, pH 7.0). The buffer is sub-sequently removed, and the cells are rapidly covered twice with the choline chloride wash buffer (120 mM choline chloride, 15 mM PIPES/tris, 0.1 mM ouabain, 1 mM MgCl₂, 2 mM CaCl₂, pH 7.4) and filtered off with suction. The cells are subsequently covered with the choline chloride charging buffer (120 mM choline chloride, 15 mM PIPES/tris, 0.1 mM PIPES/tris, 0.1 mM ouabain, 1 mM MgCl₂, 2 mM CaCl₂, pH 7.4, ²²Na^{±}(0.925 kBg/100 ml of charging buffer)) and incubated in this buffer for 6 minutes. After expiry of the incubation time, the incubation buffer is removed by suction. In order to remove extracellular radioactivity, the cells are washed rapidly four times with ice-cold phosphate-buffered saline solution (PBS). The cells are then solubilised by addition of 0.3 ml of 0.1 N NaOH per well. The cell fragment-containing solutions are transferred into scintillation tubes. Each well is then washed twice with 0.3 ml of 0.1 N NaOH, and the washing solutions are likewise introduced into the corresponding scintillation tubes. Scintillation cocktail is added to the tubes containing the cell lysate, and the radio-activity taken up into the cells is determined by determination of the β radiation.

Literature:

Counillon et al. (1993) Mol. Pharmacol. 44: 1041-1045
Franchi et al. (1986) Proc. NatI. Acad. Sci. USA 83: 9388-9392
Sardet et al. (1989) Cell 56: 271-280
Scholz et al. (1995) Cardiovasc. Res. 29: 260-268

The examples below relate to pharmaceutical preparations:

### Example A

### Injection Vials

A solution of 100 g of an NHE-3 inhibitor of the formula I and 5 g of disodium hydrogenphosphate in 3 I of bidistilled water is adjusted to pH 6.5 using 2N hydrochloric acid, sterile filtered, transferred into injection vials, lyophilised under sterile conditions and sealed under sterile conditions. Each injection vial contains 5 mg of active ingredient.

### Example B

### Suppositories

A mixture of 20 g of an NHE-3 inhibitor of the formula I is melted with 100 g of soya lecithin and 1400 g of cocoa butter, poured into moulds and allowed to cool. Each suppository contains 20 mg of active ingredient.

### Example C

### Solution

A solution is prepared from 1 g of an NHE-3 inhibitor of the formula I, 9.38 g of NaH₂PO₄.2 H₂O, 28.48 g of Na₂HPO₄.12 H₂O and 0.1 g of benzalkonium chloride in 940 ml of bidistilled water. The pH is adjusted to 6.8, and the solution is made up to 1 I and sterilised by irradiation. This solution can be used in the form of eye drops.

### Example D

### Ointment

500 mg of an NHE-3 inhibitor of the formula I are mixed with 99.5 g of Vaseline under aseptic conditions.

### Example E

### Tablets

A mixture of 1 kg of an NHE-3 inhibitor of the formula I, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### Example F

### Coated Tablets

Tablets are pressed analogously to Example E and subsequently coated in a conventional manner with a coating of sucrose, potato starch, talc, tragacanth and dye.

### Example G

### Capsules

2 kg of an NHE-3 inhibitor of the formula I are introduced into hard gelatine capsules in a conventional manner in such a way that each capsule contains 20 mg of the active ingredient.

### Example H

### Ampoules

A solution of 1 kg of NHE-3 inhibitor of the formula I in 60 I of bidistilled water is sterile filtered, transferred into ampoules, lyophilised under sterile conditions and sealed under sterile conditions. Each ampoule contains 10 mg of active ingredient.

1. Compounds of the formula I 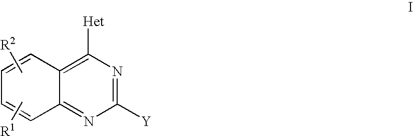
in which 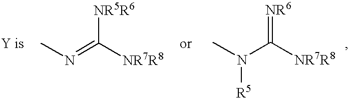
Het is a saturated, unsaturated or aromatic heterocyclic radical which is unsubstituted or monosubstituted or polysubstituted by R³ and/or R⁴,
R¹, R², R³
and R⁴ are each, independently of one another, H, A, OA, Hal, CF₃, CH₂CONH₂, CH₂CO₂H, CH₂CO₂A, CH₂NH₂, CH₂NA₂, CH₂NHA, CH₂OH, CH₂OA, OH, NO₂, NH₂, NHA, NA₂, NH-CO-A, NH-CO-Ph, SA, SO-A, SO₂-A, SO₂-Ph, CN, OCF₃, CO-A, CO₂H, CO₂A, CO-NH₂, CO-NHA, CO-NA₂, SO₂NH₂, SO₂NHA, SO₂NA₂, CHO, or are phenyl, benzyl or cyclohexylmethyl, each of which is unsubstituted or monosubstituted or polysubstituted by A, OH, OA, Hal, CN or CF₃, or are a heterocyclic radical which is monosubstituted or polysubstituted by A, OH, OA, Hal, CN or CF₃,
A is alkyl having 1, 2, 3, 4, 5 or 6 carbon atoms,
Hal is F, Cl, Br or I,
R⁵, R⁶, R⁷
and R⁸ are each, independently of one another, H, benzyl, allyl or another amino-protecting group, A, or phenyl, which is unsubstituted or monosubstituted or polysubstituted by A, OA, CN, Hal or CF₃, where R⁵ and R⁷, R⁵ and R⁶ and R⁷ and R⁸ may form 5-7-membered rings,
and salts, solvates and stereoisomers thereof, including mixtures thereof in all ratios, and pharmaceutically usable derivatives thereof.

2. Compounds of the formula I according to claim 1, characterised in that Het is 2- or 3-furyl, 2- or 3-thienyl, 1-, 2- or 3-pyrrolyl, in particular 1-pyrrolyl, 1-, 2, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothia-zolyl, 2-, 3- or 4-pyridyl, 2-, 4-, 5- or 6-pyrimidinyl, furthermore preferably 1,2,3-triazol-l-, -4- or -5-yl, 1,2,4-triazol-1-, -3- or -5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2- or 3-pyrrolidinyl, 1-, 2-, 3- or 4-piperidinyl, 2-, 3- or 4-morpholinyl, 1,4-dioxanyl, 1,3-dioxan-2-, -4- or -5-yl or 1-, 2- or 3-piperazinyl, or is selected from the following group:

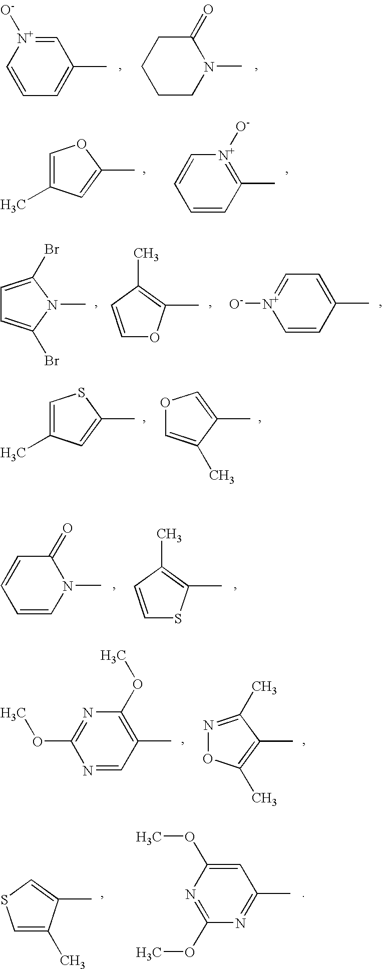

3. Compounds of the formula I according to claim 1, characterised in that R¹, R², R³ and R⁴, independently of one another, are H, A, OA, Hal, CF₃, CH₂CONH₂, CH₂CO₂H, CH₂CO₂A, CH₂NH₂, OH, NO₂, NH₂, NHA, NA₂ or NH-CO-A.

4. Compounds of the formula I according to claim 1, characterised in that R⁵ and R⁷ is simultaneously H, while R⁶ or R⁸ is H or A.

5. Compounds of the formulae IA, IB and IC: 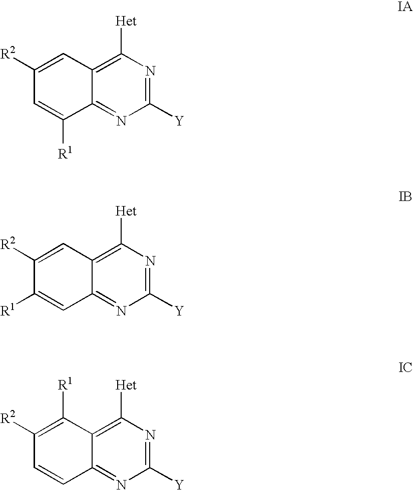
in which R¹, R², Het and Y are as defined in claim 1.

6. Compounds of the formula IA, EB and IC according to claim 5, in which R² is Cl.

7. Compounds of the formulae I1 to I14 and salts and solvates thereof:

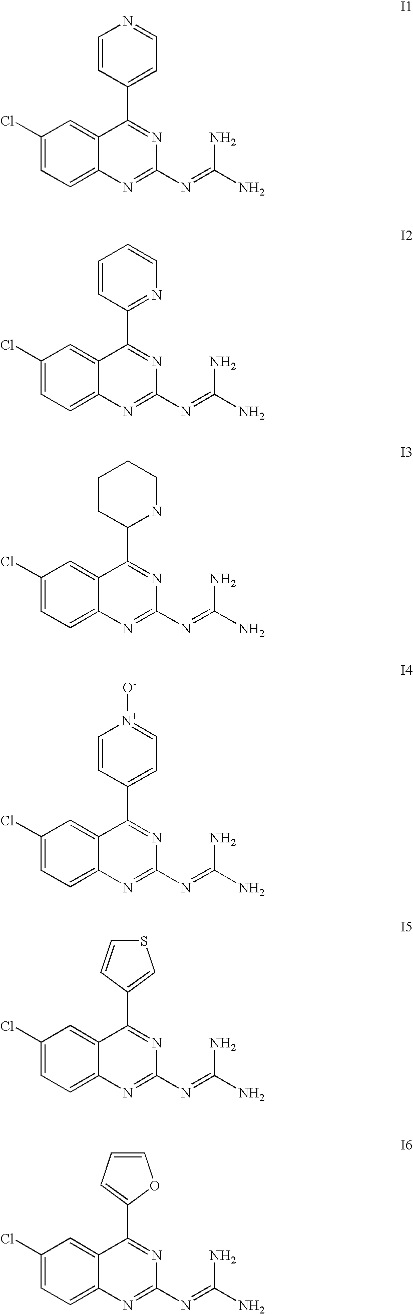

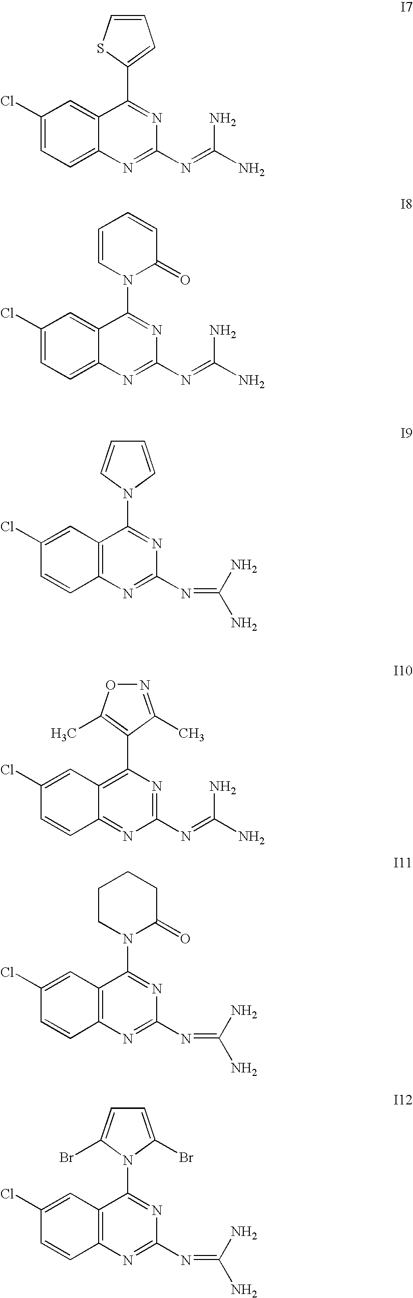

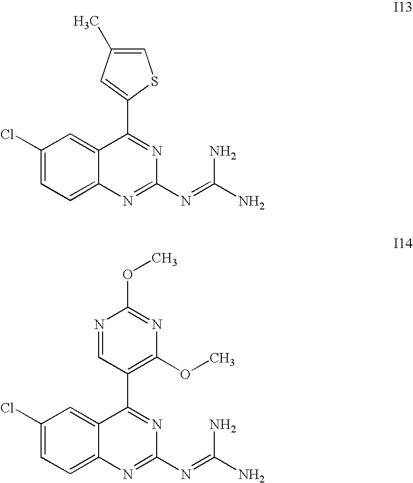

8. Compounds of the formula I according to claim 1 and salts and/or solvates thereof as NHE 3 inhibitors.

9. Compounds of the formula I according to and physiologically acceptable salts and/or solvates thereof for use in combating diseases.

10. Use of compounds of the formula I according to claim 1 and/or physiologically acceptable salts or solvates thereof for the preparation of a medicament.

11. Use of compounds of the formula I according to claim 1 and/or physiologically acceptable salts and/or solvates thereof for the preparation of a medicament for the treatment and prophylaxis of hypertonia, thrombosis, ischaemic states of the heart, of the peripheral and central nervous system and of strokes, ischaemic states of peripheral organs and extermities, and for the treatment of shock states.

12. Use of compounds of the formula I according to claim 1 and/or physiologically acceptable salts and/or solvates thereof for the preparation of a medicament for use in surgical operations and organ transplants and for the preservation and storage of transplants for surgical measures.

13. Use of compounds of the formula I according to claim 1 and/or physiologically acceptable salts and/or solvates thereof for the preparation of a medicament for the treatment and prophylaxis of diseases in which cell proliferation represents a primary or secondary cause, for the treatment or prophylaxis of disorders of fat metabolism or disturbed breathing drive.

14. Use of compounds of the formula I according to claim 1 and/or physiologically acceptable salts and/or solvates thereof for the preparation of a medicament for the treatment and prophylaxis of renal ischaemia, ischaemic intestinal diseases or for the prophylaxis of acute or chronic renal diseases.

15. Use of compounds of the formula I according to claim 1 and/or physiologically acceptable salts and/or solvates thereof for the preparation of a medicament for the treatment and prophylaxis of bacterial and parasitic diseases.

16. Pharmaceutical preparation, characterised by a content of at least one NHE-3 inhibitor according to claim 1 and/or one of its physiologically acceptable salts and/or solvates.

17. Process for the preparation of pharmaceutical preparations, characterised in that at least one compound of the formula I according to claim 1 and/or one of its physiologically acceptable salts and solvates is converted into a suitable dosage form together with at least one solid, liquid or semi-liquid excipient or adjuvant.

18. Use of compounds of the formula I according to claim 1 and/or physiologically acceptable salts and/or solvates thereof for the preparation of a medicament for the treatment and prophylaxis of diseases which are caused by increased NHE activity and/or can be influenced by a reduction in NHE activity.

19. Use of compounds of the formula I according to claim 1 and/or physiologically acceptable salts and/or solvates thereof for the preparation of a medicament for the treatment and prophylaxis of diseases or states which are caused by increased uptake of sodium ions and water in cells by organs which are undersupplied with oxygen.

20. Medicament comprising at least one compound of the formula I according to claim 1 and/or physiologically acceptable salts and solvates thereof and at least one further medicament active ingredient.

21. Set (kit) consisting of separate packs of
(a) an effective amount of a compound of the formula I according to claim 1 and/or physiologically acceptable salts and solvates thereof
and
(b) an effective amount of a further medicament active ingredient.

22. Compounds according to claim 1 as medicament active ingredients.

23. Process for the preparation of the compounds of the formula I and salts and solvates thereof, characterised in that either
(a) compounds of the formula II 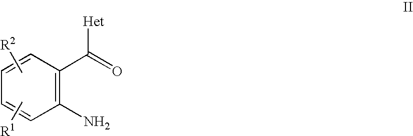
in which R¹, R² and Het are as defined in claim 1, are reacted with 1-cyanoguanidine or a correspondingly N-alkylated or N-arylated cyanoguanidine of the formula NC-Y, in which Y is as defined in claim 1,
or
(b) instead of a compound of the formula NC-Y, a compound of the formula III
HN═CX—Y III
in which X is —S-alkyl, —S-aryl, —O-alkyl or —O-aryl, is reacted with a compound of the formula II,
or
(c) compounds of the formula IV 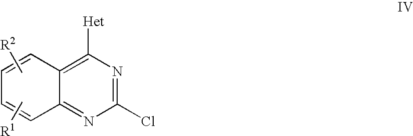
in which Het, R¹ and R² are as defined in claim 1, are reacted with a compound of the formula HY, in which Y is as defined in claim 1,
and if desired, after steps (a), (b) or (c), a basic or acidic compound of the formula I is converted into one of its salts or solvates by treatment with an acid or base.

24. Compounds of the formula II 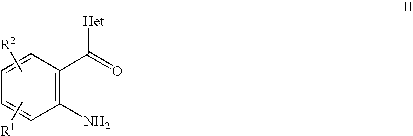
in which R¹, R² and Het are as defined in claim 1.

25. Compounds of the formula IV 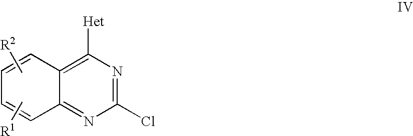
in which Het, R¹ and R² are as defined in claim 1.
